(19) United States
(12) Patent Application Publication
(10) Pub. No.: US 2026/0094675 A1
(43) Pub. Date: Apr. 2, 2026

(54) METODO PARA DETERMINAR EL GRADO DE DEGRADACION DE SOLUCIONES POLIMERICAS MEDIANTE UN MODELO REOLOGICO

(71) Applicants: YPF TECNOLOGÍA S.A., Ciudad Autónoma de Buenos Aires (AR); CONSEJO NACIONAL DE INVESTIGACIONES CIENTÍFICAS Y TÉCNICAS (CONICET), Ciudad Autónoma de Buenos Aires (AR); UNIVERSIDAD DE BUENOS AIRES, Ciudad Autónoma de Buenos Aires (AR)

(72) Inventors: Horacio Pedro BURBRIDGE, Benavidez (AR); Guillermo Osvaldo ARTANA, Ciudad Autónoma de Buenos Aires (AR)

(21) Appl. No.: 19/345,914

(22) Filed: Sep. 30, 2025

Related U.S. Application Data
(60) Provisional application No. 63/701,709, filed on Oct. 1, 2024.

(51) Int. Cl. G16C 20/10; G06F 30/28; G06F 111/10; G06F 113/08

(52) U.S. Cl. CPC G16C 20/10; G06F 30/28; G06F 2111/10; G06F 2113/08

(57) ABSTRACT

A method for determining the degree of degradation of a polymer solution comprising a polymer dissolved in water with a certain salinity and used in an enhanced oil recovery (EOR) process, said method comprising the following steps: determining the critical deformation rate s_{cr} and the dimensionless parameter Υ; and comparing the effective local deformation rates occurring in the flow within a given flow device with the critical deformation rate s_{cr} and the parameter Υ so as to determine whether or not degradation occurs in any region of the flow in a computational fluid dynamics simulation.

### FIELD OF THE INVENTION

The present invention relates to rheological models of polymer flows that may undergo mechanical degradation. More precisely, the present invention is related to a method for determining the degree of degradation of polymer solutions by means of a rheological model that is implemented in computational fluid dynamics (CFD) simulations.

### BACKGROUND OF THE INVENTION

The injection of polymer solutions into reservoirs aims to increase crude oil production by pushing it from the reservoir toward a production well. The viscosity of the solution injected into the reservoir is a key parameter. The polymer molecules used have a molecular weight higher than 106 g/mol and are very sensitive to mechanical degradation.

Due to the high injection flow rates, polymer molecules may break, losing their physical and chemical properties, in particular viscosity. This decrease in viscosity leads to a loss of efficiency in crude oil recovery.

All mature reservoirs are susceptible to being exploited through EOR (Enhanced Oil Recovery) technologies. The correct determination of the effective viscosity in polymer injection by means of perforations for each injection well is of great importance, since it will help optimize polymer injection by layer and maximize enhanced oil recovery.

Computational Fluid Dynamics (CFD) software is available on the market; however, none of them are known to consider the possibility of simulating the mechanical degradation of polymers as a consequence of the high deformation rates they may experience in fittings and conduits through which they are transported.

There are studies on the shear stability of polymers used in EOR that provide data on their behavior when subjected to degradation. In particular, in the publication “Impact of Polymer Mechanical Degradation on Shear and Extensional Viscosities: Towards Better Injectivity Forecasts in Polymer Flooding Operations”, by A. Dupas et al., the degradation of polymers passing through a capillary tube of 125 mm diameter is experimentally studied. There, degradation and viscosity curves are presented as a function of shear rate for different commercial polymers.

Another experimental study presenting degradation results on viscosity and extensional viscosity is the publication “Laminar and Turbulent Flow of Dilute Polymer Solutions in Smooth and Rough Pipes” by K. Wójs, which shows that certain conditions that do not produce significant changes in viscosity may nevertheless cause changes in extensional viscosity.

The constraints that polymer rheology imposes on production equipment are studied in the reference “Turbulent drag reduction by polymer additives: Fundamentals and recent advances” by L. Xi. The main conclusion of this work is that the viscoelastic nature of polymers has a marked influence on injection, production, and the equipment used. The response of the polymer to vibrations in flow systems tends to produce reductions in equipment efficiency and a decrease in its life cycle.

CFD is a very powerful simulation tool that makes it possible to describe the flow inside a piece of equipment and to visualize how the fluid behaves as it moves within the equipment. The use of CFD enables the design of fittings such as valves, pumps, and zones that present some particular feature in a flow line, such as elbows or reductions. Most CFD software, such as ANSYS Fluent, allows for consideration of the non-Newtonian rheology of the fluids involved in the case of laminar flows. However, the rheological models available in CFD software do not allow consideration of the effect that the “history of stress application and deformation rates” has on the subsequent behavior of the fluid, that is, whether it degrades or not, and if it does, to what extent and how that affects the downstream flow once degraded.

Consequently, there is a need for a rheological model that allows determination of the degree of mechanical degradation in polymer solutions used in enhanced oil recovery processes.

### BRIEF DESCRIPTION OF THE INVENTION

Based on the foregoing considerations, the present invention provides a method for determining the degree of degradation of a polymer solution comprising a polymer, wherein said method makes it possible to determine, through the implementation of a rheological model, whether mechanical degradation of the polymer solution will occur according to the flow fittings (e.g., valves, mandrels, reductions, expansions, elbows, etc.) used in an enhanced oil recovery process that may cause mechanical degradation. In this way, by knowing the degree of degradation of the polymer solution, it is possible to act accordingly to optimize the enhanced oil recovery process, thereby improving crude oil production efficiency. Additionally, the developed rheological model can be implemented in computational fluid dynamics software known in the state of the art.

Accordingly, it is an object of the present invention to provide a method for determining the degree of degradation of a polymer solution comprising a polymer dissolved in water with a certain salinity value and used in an enhanced oil recovery (EOR) process, said method comprising the following steps:
a) determining the critical deformation rate s_{cr} and the dimensionless parameter Υ, which is a critical value related to the transit time of the solution particles through regions where deformation rates could cause degradation, in order to determine the degree of degradation of the polymer solution based on these two critical parameters; and
b) comparing the effective local deformation rates occurring in the flow within a given flow fitting with the critical deformation rate s_{cr} and the parameter Υ, in order to determine whether degradation occurs in any region of the flow in a computational fluid dynamics simulation.

In one embodiment of the method of the present invention, prior to step a), the rheology of the non-degraded solution and the fully degraded solution is determined, wherein said rheology, both of the non-degraded solution and of the fully degraded solution, is determined by the equation:

$$η = {η}_{∞} + ( {η}_{0} - {η}_{∞} ) {( 1 + {( λ \dot{γ} )}^{a} )}^{\frac{( n - 1 )}{a}}$$

where η is the viscosity at a deformation rate \dot{Y}, η_{∞}, is defined as the viscosity at an infinite shear rate, η₀ is the viscosity at zero shear rate, λ is a shear relaxation time or characteristic shear time, n is the power-law index, and a is a dimensionless parameter describing the transition from the zero-shear-rate region to the power-law region.

In one embodiment of the method of the present invention, the critical deformation rate s_{cr} in step a) is obtained by conducting laboratory tests with the polymer solution through the following substeps:
a-i) determining a principal eigenvalue field of the deformation rate tensor, selecting from said eigenvalues the value that is positive and has the highest magnitude s_{I} (first principal eigenvalue of the rate (or velocity) of deformation tensor);
a-ii) determining the streamlines of the flow;
a-iii) determining an extreme streamline that carries the degraded solution; and
a-iv) determining the maximum principal eigenvalues along that line that produces degradation; and
a-v) assigning a maximum value of the maximum principal eigenvalues as the critical deformation rate s_{cr}.

In one embodiment of the method of the present invention, the simulation comprises calculating the first principal eigenvalue of the tensor s_{I} and the local rotation period tₜᵣₐₙₛ, point by point within the flow, and comparing the s_{I} and t_{w} values respectively with the critical values s_{cr} and Υ, wherein degradation generally occurs when the first principal eigenvalue of the deformation rate tensor s_{I} exceeds the critical value s_{cr}, that is:

$${s}_{I} \geq {s}_{c r}$$

and, at the same time:

$${s}_{I} {t}_{w} \geq γ .$$

In another embodiment of the method of the present invention, the simulation comprises calculating the first principal eigenvalue of the tensor s_{I} and the local transit time tₜᵣₐₙₛ point by point within the flow, and comparing the s_{I} and tₜᵣₐₙₛ values respectively with the critical valuess_{cr} and Υ, wherein degradation generally occurs when the first principal eigenvalue of the deformation rate tensor sᵢ s_{I} exceeds the critical value s_{cr}, that is:

$${s}_{I} \geq {s}_{c r}$$

and, at the same time:

$${s}_{I} {t}_{rans} \geq γ .$$

In this way, if the critical values s_{cr} and Υ are known in the simulation, it can be determined, as indicated in paragraphs [0015] and [0016], whether degradation occurs in any region of the simulation domain. To determine these parameters s_{cr} and Υ, tests were carried out for various solutions using two experimental setups, as indicated later in the detailed description of the invention. Typical expected ranges for these parameters were thus determined.

As indicated later in the detailed description, from the aforementioned tests it was found that conservative values of Y range between 1.2 and 2.5. For typical field cases, it is recommended to use Y equal to 2.5 as an initial tentative value. It can then be decreased, in which case some increase in degradation observed in the simulation would be expected.

As indicated later in the detailed description, the values of the critical deformation rate s_{cr} depend on the Deborah number N_{De}. For field cases, this number is typically greater than unity; therefore, in such cases, conservative values are usually in the range 500<s_{cr}<800. However, at typical field injection flow rates (on the order of 100 m³/day), it is recommended to adopt the higher end of the range, s_{cr}=800. For the purpose of testing model sensitivity, experience gained from simulations carried out during the development of the present invention has shown that in actual field cases, the value of this critical deformation rate may be set as high as s_{cr}=3000, and it is not advisable to exceed this value.

As also indicated later in the detailed description. For laboratory cases where the N_{De} number may typically be less than unity, it may be considered that 30,000<s_{cr}<70,000, and Table 7 of the present application may be used as a reference depending on the flow rate and the type of solution.

Thus, by implementing the comparisons indicated in [0015] and [0016] through user-defined routines in CFD simulators, and based on the range information for critical values indicated in [0017] to [0018], simulations may be carried out using typical values within these ranges, depending on the polymer solution in question, even without the need to conduct any prior experimental tests.

An alternative approach, which requires prior testing and simulations to obtain the critical parameters s_{cr} and Υ, consists of determining the degraded fraction of the polymer solution according to the methodology described in the articles by E. Perez et al., Sci Reports, 2024; E. Perez et al., Journal of Petroleum Science and Engineering, 2022; E. Perez et al., SPE 2025 in Press. This methodology requires only the determination, at a single temperature, of the viscosity value at a single shear rate of the original and degraded polymer solution after capillary passage. The viscosity measurement can be performed with traditional viscometers such as the Brookfield.

Thus, before performing the simulation, it would be necessary to conduct a single test using a Brookfield viscometer to determine one point of the degraded rheological curve. Then, knowing the degraded fraction and through numerical simulations for the geometry of the capillary system considered and for the flow rate at which the test was performed, the value of s_{cr} can be established as indicated later in the detailed description.

### BRIEF DESCRIPTION OF THE FIGURES

FIG. 1 shows a schematic representation of a flow situation with a contraction in a fitting.

FIG. 2 shows a schematic representation of the second experimental setup used.

FIG. 3 shows the rheologies obtained in the mechanical degradation tests with AN125 in CS water.

FIG. 4 shows the rheologies obtained in the mechanical degradation tests with AN125 in DB water.

FIG. 5 shows the rheologies obtained in the mechanical degradation tests with FP 3631s in CS water.

FIG. 6 shows the rheologies obtained in the mechanical degradation tests with FP 3631s in DB water.

### DETAILED DESCRIPTION OF THE INVENTION

Aqueous solutions of partially hydrolyzed polyacrylamides (HPAM) are non-Newtonian fluids that, within the shear rate range between 0.01 s⁻¹ and 500 s⁻¹ (and even up to 1000 s⁻¹), exhibit a viscosity that follows the Carreau-Yasuda rheological model, established by the following equation:

$$η = {η}_{∞} + ( {η}_{0} - {η}_{∞} ) {( 1 + {( λ \dot{γ} )}^{a} )}^{\frac{( n - 1 )}{a}} \qquad ( 1 )$$

where η is the viscosity at a deformation rate \dot{Y}, η_{∞} is defined as the viscosity at an infinite shear (or cut) rate, η₀ is the viscosity at zero shear rate, λ is a shear relaxation time or characteristic shear time, n is the power-law index, and a is a dimensionless parameter that describes the transition from the zero-shear-rate region to the power-law region.

In a unidirectional flow, \dot{Y} is typically the shear rate; however, in a more general way, the deformation rate can be defined, from the second invariant of the deformation rate tensor, as follows:

$$\dot{γ} = \sqrt{2 \overline{\overline{s}} : \overline{\overline{s}}}$$

$$[ \overline{\overline{s}} ] = [ \frac{1}{2} ( \frac{\partial {u}_{i}}{\partial {x}_{j}} + \frac{\partial {u}_{j}}{\partial {x}_{i}} ) ]$$

where the uᵢ correspond to the three components of the velocity vector at each point of the domain, with i equal to 1, 2, or 3 (i.e., u₁, u₂, u₃), and uⱼ) are exactly the same, but another subscript is used to indicate the possible combinations with j equal to 1, 2, or 3. The tensor s̅ is a second-order tensor with nine components, constructed by taking combinations with i=1, 2, or 3 and j=1, 2, or 3. The xᵢ and xⱼ are the coordinates, with indices also varying from 1 to 3. Since the indexes do not repeat within a single term, Einstein's summation convention does not apply; thus, all nine possible combinations must be constructed, resulting in a 3×3 matrix representing the nine components of the deformation rate tensor.

Depending on the polymer concentration, molecular weight, water salinity, pH, and temperature, the parameters η_{∞}, η₀, λ and n in equation (1) can vary significantly, hence the importance of rheological characterization of these systems under the specific field and reservoir conditions. However, in equation (1), the value of the coefficient α is taken as 2, while the value of η_{∞} is adopted as equal to the solvent viscosity ηₛ (e.g., water). This last degree of freedom is usually restricted by assuming that the viscosity at infinite shear rate η_{∞} is equal to the solvent viscosity ηₛ in equation (1), without introducing significant error.

When aqueous HPAM solutions are subjected to high deformation rates (\dot{Y}), the long-chain polymer molecules may break, resulting in a decrease in molecular weight—a phenomenon known as “mechanical degradation” of the polymer. Depending on the deformation rate (\dot{Y}) and the exposure time to these stresses, the molecular weight may decrease to the point that the solution becomes a Newtonian fluid with a viscosity practically equal to that of the solvent (water). However, if only the first chain scission is considered, the phenomenon implies a change in the Carreau-Yasuda curve describing its behavior to another Carreau-Yasuda curve with lower viscosity values, but without fully flattening as in the Newtonian case.

It should be noted that when the chain breaks, its characteristics change; therefore, to produce a new breakage of the two resulting polymer chains, even higher deformation rates are required. Since the method of the present invention involves the flow of polymer solutions through valves and flow fittings, once the solution passes through the restriction, it is not expected to undergo an even greater restriction downstream that could produce further chain breakage from the first degradation. Therefore, the method of the present invention considers only the first chain scission for the rheological degradation model used in numerical simulations.

The intrinsic viscosity [η] of a solution can be considered as a measure of the volume of a polymer chain immersed in a solvent when the distance between molecules is very large (dilute solution). It is a quantity that depends solely on the polymer and the solvent (water) used to prepare the solution. By definition, it is the value of the ratio between the specific viscosity and the concentration (of the polymer in the solution) in the limit as concentration and shear rate tend to zero.

There exists a relationship between the intrinsic viscosity [η] of the polymer solution and the viscosity value at zero shear rate η₀. This relationship can be approximated by an expression of the form

$$\frac{{η}_{0} - {η}_{S}}{{η}_{s}} = C [ η ] + {{a}_{1} ( C [ η ] )}^{1 , 31} + {{a}_{2} ( C [ η ] )}^{3 , 93} \qquad ( 2 )$$

where C denotes the polymer concentration in the solution. The values of the prefactors a₁ and a₂, obtained from fitting experimental data for HPAM in the work of Jouenne and Levache, “Universal viscosifying behavior of acrylamide-based polymers used in enhanced oil recovery,” are 0.667 and 0.028, respectively. In this way, from equation (2), the value of η₀ for use in equation (1) can be obtained.

For the value of the exponent n in the Carreau law, the same author proposes the following expression:

$$n = 1 - ( 0.796 - 0.687 {e}^{- 0.0 \; 59 C [ η ]} ) \qquad ( 3 )$$

where, from said equation (3), the value of n for equation (1) can be obtained.

Meanwhile, for the parameter λ of the Carreau law, the same author proposes the following expression:

$$λ = {λ}_{d} ( 1 + 0.4 {( C [ η ] )}^{2.4} ) \qquad ( 4 )$$

where λ_{d} is the relaxation time in the dilute regime and is approximately equal to:

$${λ}_{d} ∼ 1.4 \; 74 \frac{[ η ] {η}_{s} {M}_{w}}{T}$$

where M_{w} represents the molecular weight of the polymer and T the temperature of the solution. Thus, from said equation (4), the value of λ for equation (1) can be obtained.

When the polymeric solution is composed of binary mixtures of polymers with different molecular weights (for example, a polymer that has undergone mechanical degradation together with another polymer that has not been degraded), the intrinsic viscosity of the mixture takes a value that is calculated from:

$$η = \frac{{{\sum }_{i = 1} [ η ]}_{i} {C}_{i}}{{\sum }_{i = 1} {C}_{i}} \qquad ( 5 )$$

where Cᵢ indicates the concentration of polymer i. In particular, the subscript indicates the possibility of having different polymers, which is the situation that arises when there is a single polymer, but then some molecules are mechanically degraded and others are not. The degraded molecules can be considered as a new polymer with another molecular weight. Therefore, for practical purposes, it can be considered that, after degradation, there are two solutions with two different polymers and with two different concentrations, since the amount of degraded molecules does not necessarily have to be equal to the amount of non-degraded molecules.

The intrinsic viscosity of the solute is related to its molecular weight through the Mark-Houwink relationship:

$$[ η ] = K {M}_{w}^{α} \qquad ( 6 )$$

where the constants K and α depend on the polymer-solvent system considered. The theoretical value of the coefficient α for good solvents is 0.764, and in general a takes values in the range from 0.5 to 0.8, while for HPAM K≅1.66.

The stretching produced by the flow causes chain rupture and consequently a decrease in molecular weight. Thus, if the entire solution is degraded, the intrinsic viscosity will modify its viscosity as the molecular weight of the chain changes according to the Mark-Houwink relationship. If, as a consequence of the flow, only part of the solution undergoes mechanical degradation, the intrinsic viscosity of the mixture will be modified according to equation (5), taking into account the degraded and non-degraded fractions.

The degradation of the polymeric solution as a consequence of the action of the flow depends on the rate of deformation (it is mainly the elongation rates that cause rupture) to which the chain is subjected. The critical value required to cause the rupture of a chain depends on its molecular weight according to a law such as the following:

$${s}_{c r} ∼ {M}_{w}^{- c} \qquad ( 7 )$$

where the value of the constant c is usually between 1 and 2.

Law (7) shows that, for a given flow configuration, and consequently for a given rate of deformation (elongation), the molecules susceptible to rupture are those that have a high molecular weight. As the chains break, their molecular weight decreases, and consequently, to undergo further ruptures they will require higher elongation rates.

In the mechanical degradation of polymers, several characteristic times of importance can be defined; however, the method of the present invention uses three of them: the Zimm relaxation time (t_{z}), the transit time (tₜᵣₐₙₛ), and the rotation period (t_{ω} of a particle (ω being the vorticity of the particle). In particular, these times are defined as follows:

$${t}_{z} = \frac{[ η ] {η}_{s} {M}_{w}}{R T} \qquad ( 8 )$$

where R is the universal gas constant and T the temperature of the fluid;

$${t}_{trans} = \frac{{ϕ}_{c}^{3}}{Q} \qquad ( 9 )$$

where Q is the volumetric flow rate and φ_{c} is the critical passage diameter; and

$${t}_{ω} = \frac{1}{ω} \qquad ( 10 )$$

$$wherein$$

$$\overline{ω} = rot \overline{V}$$

$$\overline{V} = {u}_{i} {{e}^{⋁}}_{i}$$

where the vector V̅ is the velocity of the particle, where the uᵢ are the velocity components and the ěj are the orthonormal basis vectors of a Cartesian triad, and Einstein's summation convention is applied with i equal to 1, 2, or 3, that is:

$$\overline{V} = {u}_{1} {{e}^{⌣}}_{1} + {u}_{2} {{e}^{⌣}}_{2} + {u}_{3} {{e}^{⌣}}_{3}$$

It should be noted that the transit time is a reference magnitude based on the equivalent diameter in the restriction; the Zimm relaxation time is a property of the solution that depends on the temperature; and the rotation time or period is a magnitude that depends, in general, on position and time (that is, it is a flow variable).

Based on the previously defined times and elongation rates, the dimensionless Weissenberg number (Wᵢ) and Deborah number (N_{De}) can be defined. In particular, these dimensionless numbers are defined as follows:

$${W}_{i} = {s}_{I} t \qquad ( 11 )$$

$${N}_{De} = \frac{{t}_{z}}{{t}_{trans}} \qquad ( 12 )$$

where, in expression (11), s_{I} is the first principal eigenvalue of the rate (or velocity) of deformation tensor, and t may be taken equal to tₜᵣₐₙₛ or also t equal to t_{ω}.

It should be understood that the Deborah number quantifies the ratio between the Zimm relaxation time and the transit time of a molecule in regions where degradation may occur (regions of high elongation rate). In the case of laminar flows, two different rupture regimes are identified depending on the values adopted by this parameter. For N_{De}>>1 (fast passage), the molecule fully uncoils and then breaks. For N_{De}<<1 (slow passage), the polymer molecule partially uncoils and then cleaves.

For dilute solutions in laminar regime, this results in the critical elongation rates being s_{cr}˜M_{w}⁻¹ when N_{De}>>1 and s_{cr}˜M_{w}⁻² when N_{De}<<1. Thus, the same solution may exhibit different values of critical elongation rate depending on the passage time through the degradation region. To produce rupture, slow passages require higher elongation rates than fast passages. In other words, slow passages through zones of high elongation rate can be more easily withstood by the molecules without fission.

In one embodiment of the method of the present invention, it makes use of the following rheological model that accounts for mechanical degradation for CFD numerical simulation. The rheology of the undegraded solution is assumed to be known, of the Carreau-Yasuda law type; that is, the coefficients η_{∞}, η₀, λ, n and a of the Carreau-Yasuda law given by expression (1) are known. The rheology of the completely degraded solution (all fluid degraded) after the first rupture breakage of the polymer chains is also assumed to be known; that is, the corresponding coefficients η_{∞}, η₀, λ, n and a. are also known.

The variables considered are the volumetric flow rate (Q); the critical area (A*), which corresponds to the smallest passage area, and where, if the passage occurs through several holes located in the same section or close to each other, this area is obtained by summing the passage areas of all the holes; the equivalent diameter (φ_{c}), the mean velocity (V*) in the critical section, and the equivalent viscosity (η_{eq}). In particular, the equivalent diameter and the mean velocity are obtained from the following expressions:

$${ϕ}_{c} = \sqrt{\frac{4}{π} {A}^{*}}$$

$${V}^{*} = \frac{Q}{{A}^{*}}$$

while the equivalent viscosity is obtained from the Carreau-Yasuda curve of the undegraded solution by using an equivalent shear rate value calculated as follows:

$${\dot{γ}}_{eq} = \frac{{V}^{*}}{{ϕ}_{c}} that is ,$$

$${η}_{eq} = {η}_{∞} + ( {η}_{0} - {η}_{∞} ) {( 1 + {\dot{γ}}_{eq}^{a} )}^{\frac{( n - 1 )}{a}}$$

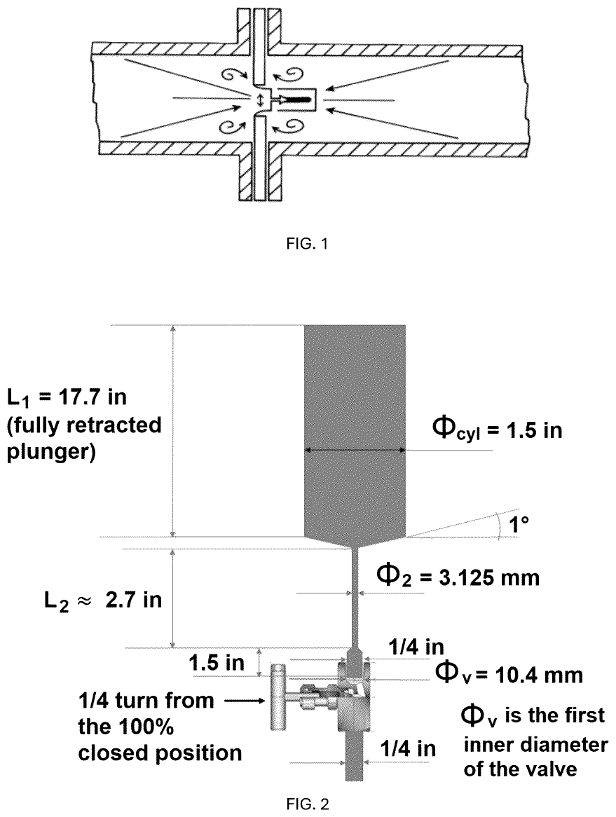

It should be noted that the above expressions take into account a situation of flow of the polymeric solution with a contraction as shown in FIG. 1, and that, although the actual flow fittings (for example, valves, mandrels, or others) will not, in general, have the geometry shown in said FIG. 1, in general there will exist zones of abrupt enlargement or narrowing or sharp bends, which are precisely what are expected to cause degradation. In other words, although the geometry of FIG. 1 is that of an orifice plate and is not the same as the geometry of a valve, mandrel, etc., the situation is nevertheless similar or analogous, because in all cases there is a contraction or flow disturbance that can potentially produce mechanical degradation.

The rheological model used in the method of the present invention is based on the assumption that, when passing through the critical section, breakage of the polymer chains (first breakage) will occur in some areas in the vicinity of the critical section. In particular, said rheological model takes into consideration that the polymer has a monodisperse molecular weight distribution; that chain rupture generates two new equal chains, each half the length of the original; and that the flow of the polymer solution is laminar when reference global parameters (such as the valve inlet diameter) are considered for determining a global Reynolds number. Additionally, said rheological model considers that turbulent flow situations may occur in regions near the flow restriction; however, when polymer concentrations are significant (>100 ppm), injection flow rates are typically laminar, and if turbulence exists, it is localized, with a rapid decay of such turbulence to a laminar regime. Thus, the rheological model assumes laminar flow, understanding that if some localized instabilities appear, the same decay rapidly and have no significant effects on degradation.

In the rheological model of the method of the present invention, degradation is generally considered to occur when the first principal eigenvalue of the deformation rate tensor, s_{I} exceeds a certain critical value, that is:

$${s}_{I} \geq {s}_{cr}$$

and, at the same time:

$${s}_{I} t \geq ϒ$$

where t represents the residence time, indicating how long the polymer molecule is subjected to high deformation rates that can cause degradation, and the value Υ is a dimensionless number associated with the way t is defined. For the latter, t may be taken as t_{ω}, thus:

$${s}_{I} {t}_{ω} \geq ϒ$$

It should be noted that, in the previous expression, t could also be taken as tₜᵣₐₙₛ.

It should also be noted that Υ is also a critical value of a form of the Weissenberg number given by s_{I}, t_{ω}. It is also important to mention that this last condition is equivalent to assuming that:

$${P}_{e} {t}^{+} = {s}_{I} {t}_{ω} \geq ϒ$$

where Pₑ is the dimensionless Peclet number and t⁺ is the Rognin number, which is a ratio of characteristic times.

Considering the expected characteristic values of the critical deformation rate for HPAM (on the order of 10⁴ s⁻¹) and the cases considered by Rognin and collaborators in “A multiscale model for the rupture of linear polymers in strong flows,” where Υ is approximately 4 (Y˜4), the residence times in the region where the stretching rate exceeds the critical deformation rate value must be greater than a value close to milliseconds.

Once this model has been established and the experimental rheological curves obtained, it becomes evident that, for the purposes of simulation, the composition of the polymer solution (ppm, water, polymer brand) is not relevant, but only the following data: (i) the rheology of the undegraded solution (Carreau-Yasuda curve); (ii) the molecular weight of the polymer used (ensuring that the considerations of the model described above are satisfied); (iii) the rheology of the degraded solution (Carreau-Yasuda curve for 100% degradation of the fluid corresponding to the first breakage of the polymer chains); and (iv) the degradation criteria given by the values of s_{cr} and Υ, which must be determined.

It should be noted that the rheologies (Carreau-Yasuda curve according to equation (1)), both for the undegraded solution and for the solution after 100% degradation of the fluid corresponding to the first breakage of the polymer chains, are obtained experimentally. Once these data are known, the critical stretching rates s_{cr} and the coefficient Υ for the degradation criterion can be determined and then, by implementing the developed routines or codes, CFD simulations can be performed in order to determine whether degradation occurs in any region of the flow by comparing the local effective deformation rates occurring in the flow with the critical deformation rate s_{cr} and the parameter Υ.

It should be noted that one of the advantages of the method of the present invention lies in the degradation criterion and the methodology or protocol for determining the critical values, together with the implementation to integrate it into a CFD.

### Tests

The polymers tested for the present invention exhibit a molecular weight distribution centered around a mean value. Under certain flow conditions, mechanical degradation modifies this distribution due to molecular rupture. For dilute solutions, rupture generally occurs approximately at the midpoint of the chain length. In cases of successive ruptures, this latter value could be further reduced to half its previous value. However, the occurrence of successive ruptures requires subjecting the solution to a much higher stretching rate than that causing the first rupture.

In the case of single ruptures, the degraded fraction tends to generate a solution centered at a value equal to half the initial mean molecular weight. If only a fraction of the solution is degraded, two peaks would coexist in the distribution: one corresponding to the undegraded solution and another corresponding to the degraded solution. The degree of degradation can be estimated by analyzing molecular weight distribution curves.

In a first experimental setup, it is required to determine the Carreau curve for both the undegraded solution and the degraded solution (first breakage), in order to adequately model their behavior under different flow conditions. In this first experimental setup, the method described in the publication “Shear Stability of EOR Polymers” by A. Zaitoun et al. was used, which basically consists of pressurizing a vessel by means of a flow-controlled pump and forcing the polymer solution to pass through a system ending with a capillary tube of known internal diameter. The pressure is kept constant, and the capillary diameter is gradually reduced to achieve higher shear rates.

It is at the contraction occurring at the capillary tube inlet where the mechanical degradation of the polymer mainly takes place. The stretching rates, or extensional deformation rates, are those that have the greatest impact on chain breakage. The matrix of tests performed was as follows:

TABLE 1
Test matrix with the first experimental setup.

| | | | | | | | | 200 | 200 | 200 |
|---|---|---|---|---|---|---|---|---|---|---|
| | | | 0 | 10 | 50 | 100 | 150 | ml/min | ml/min | ml/min |
| Polymer | Water | Ppm | ml/min | ml/min | ml/min | ml/min | ml/min | 1 Passage | 2 Passages | 3 Passages |
| [symbol] | [symbol] | [symbol] | ✓ | ✓ | ✓ | ✓ | ✓ | ✓ | | |
| | | [symbol] | | | | | | | | ✓ |
| | | [symbol] | ✓ | ✓ | ✓ | ✓ | ✓ | ✓ | | |
| | | [symbol] | | | | | | | | ✓ |
| | | [symbol] | ✓ | ✓ | ✓ | ✓ | ✓ | ✓ | | |
| | | [symbol] | ✓ | ✓ | ✓ | ✓ | ✓ | ✓ | | |
| | | [symbol] | ✓ | ✓ | | ✓ | ✓ | ✓ | ✓ | ✓ |
| | [symbol] | [symbol] | ✓ | ✓ | ✓ | ✓ | ✓ | ✓ | | |
| | | [symbol] | | | | | | | | ✓ |
| | | [symbol] | ✓ | ✓ | ✓ | ✓ | ✓ | ✓ | | |
| | | [symbol] | | | | | | | | ✓ |
| | | [symbol] | ✓ | ✓ | ✓ | ✓ | ✓ | ✓ | | |
| | | [symbol] | ✓ | ✓ | ✓ | ✓ | ✓ | ✓ | | |
| | | [symbol] | | | | | | | | ✓ |
| [symbol] | [symbol] | [symbol] | ✓ | ✓ | ✓ | ✓ | ✓ | ✓ | | |
| | | [symbol] | ✓ | ✓ | ✓ | ✓ | ✓ | ✓ | | |
| | | [symbol] | ✓ | ✓ | ✓ | ✓ | ✓ | ✓ | | |
| | | [symbol] | ✓ | | | | | | | ✓ |
| | | [symbol] | ✓ | ✓ | ✓ | ✓ | ✓ | ✓ | | |
| | | [symbol] | ✓ | | | | | | | ✓ |
| | [symbol] | [symbol] | ✓ | ✓ | ✓ | ✓ | ✓ | ✓ | | |
| | | [symbol] | ✓ | ✓ | ✓ | ✓ | ✓ | ✓ | | |
| | | [symbol] | | | | | | | | ✓ |
| | | [symbol] | ✓ | ✓ | ✓ | ✓ | ✓ | ✓ | | |
| | | [symbol] | ✓ | ✓ | ✓ | ✓ | ✓ | ✓ | | |
| | | [symbol] | | | | | | | | ✓ |

[symbol]

indicates data missing or illegible when filed

For these tests, synthetic waters were used, corresponding to two brines similar to the formation waters of the mature outfields of Desfiladero Bayo and Cañadón Seco, where the injection water used in Desfiladero Bayo is characterized by low salinity, while the injection water from Cañadón Seco is among the highest in salinity. Theses synthetic waters were designated as CS water (similar to the injection water from Cañadón Seco) and DB water (similar to the injection water from Desfiladero Bayo). The CS brine contained 24,530 ppm of total dissolved solids, composed of 980 ppm of CaCl₂, 21,840 ppm of MgCl₂·6H₂O, 10 ppm of Na₂SO₄, and 21700 ppm of NaCl. Similarly, the DB brine contained 1,300 ppm of total dissolved solids, composed of 400 ppm of CaCl₂, 280 ppm of MgCl₂·6H₂O, 370 PPM 106 NaSO₄, 210 ppm of NaHCO₃ and 40 ppm of NaCl. In this way, the range of salinity extremes is covered.

To determine predictive models for the mechanical degradation of polymer solutions, two polymers were used in the tests: SNF Flopaam 3631S™ and SNF AN-125 VHM™. Both are HPAMs, i.e., high molecular weight water-soluble polymers typically used in EOR.

The rheologies of both degraded and undegraded solutions were measured, and the rheology values were found to be reliable within the shear rate range between 1 s⁻¹ and 1000 s⁻¹. The oscillations observed at the lowest shear rate values in many cases limited the ability to accurately determine the zero shear rate value for the given solution. The difficulties encountered led, in some cases, to the repetition of tests with newly prepared solutions. These repeated cases are listed in the test table with an indication in parentheses using the letter (b), while the first set is denoted with the letter (a). With the limitations described, fittings were carried out according to the Carreau law, with coefficient values detailed in the following tables.

TABLE 2
Zero-shear viscosity values (η₀).

| | | | | | | | | 200 | 200 | 200 |
|---|---|---|---|---|---|---|---|---|---|---|
| | | | 0 | 10 | 50 | 100 | 150 | ml/min | ml/min | ml/min |
| Polymer | Water | Ppm | ml/min | ml/min | ml/min | ml/min | ml/min | 1 Passage | 2 Passages | 3 Passages |
| [symbol] | [symbol] | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | [symbol] | [symbol] | | [symbol] | [symbol] | [symbol] | [symbol] | [symbol] |
| | [symbol] | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | | | | | | | | [symbol] |
| [symbol] | [symbol] | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | [symbol] | | | | | | | [symbol] |
| | | [symbol] | [symbol] | [symbol] | [symbol] | [symbol] | [symbol] | [symbol] | | |
| | | [symbol] | [symbol] | | | | | | | [symbol] |
| | [symbol] | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | | | | | | | | [symbol] |

[symbol]

indicates data missing or illegible when filed

TABLE 3
Values of coefficient n from the Carreau expression.

| | | | | | | | | 200 | 200 | 200 |
|---|---|---|---|---|---|---|---|---|---|---|
| | | | 0 | 10 | 50 | 100 | 150 | ml/min | ml/min | ml/min |
| Polymer | Water | Ppm | ml/min | ml/min | ml/min | ml/min | ml/min | 1 Passage | 2 Passages | 3 Passages |
| [symbol] | [symbol] | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | [symbol] | [symbol] | | [symbol] | [symbol] | [symbol] | [symbol] | [symbol] |
| | [symbol] | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | | | | | | | | [symbol] |
| [symbol] | [symbol] | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | [symbol] | | | | | | | [symbol] |
| | | [symbol] | [symbol] | [symbol] | [symbol] | [symbol] | [symbol] | [symbol] | | |
| | | [symbol] | [symbol] | | | | | | | [symbol] |
| | [symbol] | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | | | | | | | | [symbol] |

[symbol]

indicates data missing or illegible when filed

TABLE 4
Values of coefficient λ from the Carreau expression.

| | | | | | | | | 200 | 200 | 200 |
|---|---|---|---|---|---|---|---|---|---|---|
| | | | 0 | 10 | 50 | 100 | 150 | ml/min | ml/min | ml/min |
| Polymer | Water | Ppm | ml/min | ml/min | ml/min | ml/min | ml/min | 1 Passage | 2 Passages | 3 Passages |
| [symbol] | [symbol] | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | [symbol] | [symbol] | | [symbol] | [symbol] | [symbol] | [symbol] | [symbol] |
| | [symbol] | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | | | | | | | | [symbol] |
| [symbol] | [symbol] | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | [symbol] | | | | | | | [symbol] |
| | | [symbol] | [symbol] | [symbol] | [symbol] | [symbol] | [symbol] | [symbol] | | |
| | | [symbol] | [symbol] | | | | | | | [symbol] |
| | [symbol] | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | | | | | | | | [symbol] |
| | | [symbol] | [symbol] | [symbol] | [symbol] | [symbol] | [symbol] | [symbol] | | |
| | | [symbol] | [symbol] | [symbol] | [symbol] | [symbol] | [symbol] | [symbol] | | |
| | | [symbol] | | | | | | | | [symbol] |

[symbol]

indicates data missing or illegible when filed

As can be seen in Tables 2 to 4, as the solution undergoes degradation, the monotonic decrease in zero-shear viscosity presents inconsistencies in some cases corresponding to tests performed at lower flow rates.

Taking into account the experimental data, when available, and possible interpolations of values from the curves in the work by Jouenne and Levache mentioned previously, the rheological values of the fractions of completely degraded solutions are detailed below. For this purpose, the coefficients of the corresponding Carreau laws are indicated.

TABLE 5
Rheology of completely degraded solutions.

| | | ppm | η₀ | λ | |
|---|---|---|---|---|---|
| Polymer | Water | Concentration | mPas | s | n |
| FLOOPAM 3631 | CS | 1000 | 2.6 | 0.01 | 0.82 |
| FLOOPAM 3631 | CS | 2000 | 8.7 | 0.02 | 0.77 |
| FLOOPAM 3631 | CS | 3000 | 17.5 | 0.04 | 0.70 |
| FLOOPAM 3631 | CS | 4000 | 36.7 | 0.05 | 0.65 |
| FLOOPAM 3631 | DB | 1000 | 7.6 | 0.02 | 0.65 |
| FLOOPAM 3631 | DB | 2000 | 55.0 | 0.09 | 0.51 |
| FLOOPAM 3631 | DB | 3000 | 430 | 0.62 | 0.45 |
| FLOOPAM 3631 | DB | 4000 | 9339 | 26.3 | 0.41 |
| AN125 | CS | 1000 | 3.2 | 0.01 | 0.8 |
| AN125 | CS | 2000 | 8.1 | 0.01 | 0.73 |
| AN125 | CS | 3000 | 15.5 | 0.02 | 0.66 |
| AN125 | CS | 4000 | 28.1 | 0.02 | 0.58 |
| AN125 | DB | 1000 | 5.75 | 0.11 | 0.78 |
| AN125 | DB | 2000 | 39.3 | 0.15 | 0.62 |
| AN125 | DB | 3000 | 191.2 | 0.20 | 0.60 |
| AN125 | DB | 4000 | 52.1 | 1.74 | 0.54 |

These values must be considered in the simulations as properties advected by the degraded fluid particles. The value of coefficient α is taken as 2 in all cases, while the value of η_{∞} is adopted equal to the solvent viscosity ηₛ.

The extrapolation of these values to others not included in the table is achieved using the master curves of Jouenne and Levache, employing the corresponding intrinsic viscosity.

In addition to the tests described above to determine rheology, intrinsic viscosity measurements were carried out for the solutions of interest. It should be recalled that intrinsic viscosity is a quantity that depends solely on the polymer considered and on the water used to prepare the solution; therefore, concentration is not a relevant factor. Furthermore, once the polymer is degraded, its molecular weight changes and it must be considered as a different polymer; consequently, the intrinsic viscosity of the degraded solution is not equal to that of the undegraded solution. Therefore, for these solutions, eight different cases must be considered, as shown in the following table:

TABLE 6
Intrinsic viscosity values for the different solutions.

| | | | | Intr. Visc. |
|---|---|---|---|---|
| | | | Exp. Intr. | (Jouenne |
| | | | Visc. Y-TEC | method) |
| Polymer | Water | Degradation | l/g | l/g |
| FLOOPAM 3631 | CS | Sin | 1.46 | 1.82 |
| FLOOPAM 3631 | CS | Con | 0.82 | 0.78 |
| FLOOPAM 3631 | DB | Sin | 3.33 | 5.18 |
| FLOOPAM 3631 | DB | Con | 2.53 (2.05) | 2.06 |
| AN125 | CS | Sin | 1.2 | 1.7 |
| AN125 | CS | Con | 0.84 | 1.06 |
| AN125 | DB | Sin | 3.69 (3.52) | 2.31 |
| AN125 | DB | Con | 3.18 (2.05) | 1.27 |

The values were obtained using Cannon-Fenske viscometer No. 50×122 and are shown in the fourth column, where the values of repeated tests are indicated in parentheses. The determination of intrinsic viscosity using this type of device presents errors when the shear rates developed in the device are high. Considering the flow rates that occurred in the device during the tests performed, the shear rate in these studies was found to exceed 100 s⁻¹. It is concluded that the reported values may contain errors. These values were later corrected based on the master curves from the work of Jouenne and Levache mentioned above, which were constructed from the results of 400 experiments with partially hydrolyzed polyacrylamides similar to the Flopaam 3631 and AN-125 polymers. The corrected values are also presented in Table 6 (in the last column), where the rows indicating degraded solution correspond to a solution that has passed three times through the device's capillary tube at a flow rate of 200 mL/min to ensure complete degradation (first breakage of all molecules).

Determination of the critical degradation values s_{cr} y Υ requires experimental analysis and numerical simulation in order to make the numerical results converge with the experimental ones. First, experimental tests are carried out attempting to cover sufficiently wide ranges of Deborah numbers. In order to obtain a wide range of Deborah numbers, two different experimental setups had to be used.

The first is the same experimental setup described above, passing the previously described solutions through the capillary tube at different flow rates. The rheology of the degraded solutions was then measured, and the mass fraction of degraded solution was calculated, as explained previously. For the flow rates of these tests, considering the flow area (cross-sectional area of the capillary tube) and the transit and Zimm relaxation times, in all cases for this experimental setup the Deborah numbers result much lower than one, that is, N_{De}<<1 (slow passage). For dilute solutions in laminar regime, this results in the critical elongation rates being s_{cr}˜M_{w}⁻² when N_{De}<<1

From the results obtained with the first experimental setup to determine the critical stretching rate s_{cr} of the different polymeric solutions, the following protocol was used: i) numerical simulations of the experimental device were carried out for the flow rates used in the experiments, considering that the fluid is non-Newtonian and follows a Carreau law, where the simulations were performed using the ANSYS Fluent 19.1 software; ii) determination of the field of principal eigenvalues of the rate-of-deformation tensor (only that which is positive and has the highest value is of interest); iii) determination of the streamlines of the problem; iv) determination of the extreme streamline that transports degraded solution; v) determination of the maximum principal eigenvalues along that line that produce degradation; and vi) the maximum value is identified with the value of the critical extensional rate of the solution.

On the other hand, for flow rates typically observed in the field, and with the typical geometries of flow devices used in the field, the Deborah numbers are usually higher, i.e., N_{De}>>1 (fast passage), and for dilute solutions in laminar flow, this results in the critical stretch rates being s_{cr}˜M_{w}⁻¹ when N_{De}>>1. Hence, a second experimental setup had to be used which, under controlled laboratory conditions, would allow obtaining flows with higher Deborah numbers. This second experiment basically consists of replacing the capillary tube with a valve known to cause degradation, and determining a valve opening sufficiently restricted to obtain higher passage velocities in the critical zone, which determines higher Deborah numbers for this condition. Thus, some solutions are passed through said valve, and the rheologies and degraded mass fractions are obtained after the passage, in the same way as was done with the first experimental setup.

Then, numerical simulations are carried out, with the degradation criterion already implemented in ANSYS Fluent via a user-defined function (UDF), representing the same flow as in the experiments of the second experimental setup, seeking to adjust the values of s_{cr} and Υ in the numerical model so as to observe in the simulation a viscosity decay and a degraded solution mass fraction as close as possible to those observed in the experimental tests.

More precisely, the valve used in the second experimental setup was the ABAC VA125 valve, which is known to degrade the polymer and is therefore used with a device designed for extraction under a specific procedure to avoid such degradation. However, in the tests below, the purpose is to produce degradation in order to evaluate the model's ability to predict it, and to find the critical parameters by fitting. FIG. 2 shows a schematic diagram of the second experimental setup used.

Four rheological cases were selected, which allows concentrating the effort of obtaining experimental and simulation results on a few representative combinations of rheologies typically used in the field in EOR projects. The selected rheologies (obtained from the tests with the first experimental setup of Table 5) for the tests with the second experimental setup were: −2000 ppm Floopam 3631S in CS water, −2000 ppm Floopam 3631S in DB water, −2000 ppm AN125 in CS water, and −2000 ppm AN125 in DB water.

The device used in the second experimental setup therefore operates at constant flow rate, with pressure values inside the cylinder that depend on the pressure drop in the valve. It was therefore decided to test three different flow conditions for each solution, namely:

$${Q}_{1} = 100 \frac{ml}{min}$$

$${Q}_{2} = 150 \frac{ml}{min}$$

$${Q}_{3} = 200 \frac{ml}{min}$$

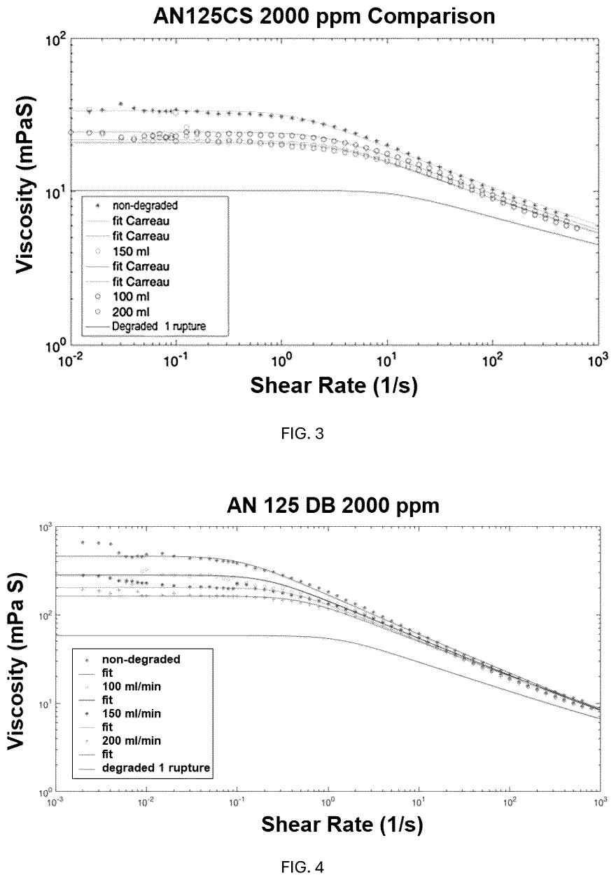

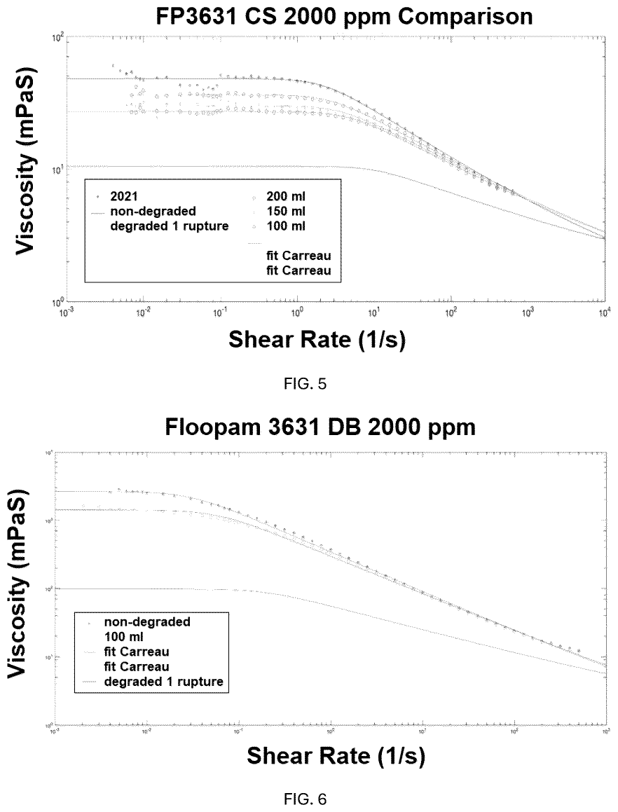

The results of the tests are shown in FIGS. 3, 4, 5, and 6. In all these figures, the curve with the lowest viscosity corresponds to the fully degraded rheology, for the first breakage, which is indicated as “degraded 1 rupture.”

The rheological values corresponding to the flow rates of 100 ml/min, 150 ml/min, and 200 ml/min are not shown in FIG. 6 for the case of 2000 ppm of the polymer Floopam 3631S in DB water. This is because the results obtained for these curves presented some inconsistencies, and therefore this case was discarded.

On the other hand, it was observed that the cases corresponding to the solutions of 2000 ppm AN125 in CS water and 2000 ppm AN125 in DB water constitute two extreme cases in terms of the viscosity levels actually injected in the field, and therefore they can be adopted as reference extreme cases.

In all cases, the Reynolds number based on the critical section was calculated according to the following equation:

$${R}_{e}^{*} = \frac{{V}^{*} {ϕ}_{c} ρ}{{η}_{eq}}$$

and the flow was always laminar within the valve restriction, with values below 1000 in all cases. Furthermore, as described above, even if the Reynolds number reached values close to 2×10⁴, for typical rheologies it would not make sense to consider a turbulent regime.

The results obtained from simulations carried out for the cases of the first experimental setup indicated in Table 4, for low Deborah numbers, are presented below.

TABLE 7
Critical deformation rate values (for different flow rates and average). Low Deborah numbers.

| | | | | | | | S_{cr} |
|---|---|---|---|---|---|---|---|
| | | | | | | | AVERAGE |
| | | | | | | 200 ml/min | (last 3 flow rates) |
| Polymer | Water | Ppm | 50 ml/min | 100 ml/min | 150 ml/min | 1 passage | S⁻¹ |
| FLOOPAM | CS | 1000(a) | s/deg | s/deg | n/a | n/a | n/a |
| 3631S | | 2000(a) | 57826 | 49031 | 47103 | 49387 | 48507 |
| | | 3000(a) | 21160 | 40144 | 43329 | 47457 | 43643 |
| | | 4000(a) | 15730 | 24828 | 31720 | 36170 | 30906 |
| | DB | 1000(a) | 55087 | 46554 | 48706 | 47314 | 47525 |
| | | 2000(a) | 23473 | 42840 | 58611 | 55103 | 52185 |
| | | 3000(a) | 23769 | 48519 | 58293 | 55147 | 53986 |
| | | 4000(a) | 23766 | 46829 | 58401 | n/a | 52615 |
| AN125 | CS | 1000(a) | non-degraded | non-degraded | 54184 | 57885 | 56035 |
| | | 2000(a) | 23486 | 42081 | 38930 | 39587 | 40199 |
| | | 3000(a) | 17172 | 24775 | 32437 | 35986 | 31066 |
| | | 4000(a) | 14751 | 23888 | 30720 | 29755 | 28121 |
| | DB | 1000(a) | 69617 | 74539 | 79452 | 56189 | 70060 |
| | | 2000(a) | 50127 | 49422 | 66143 | 45422 | 53662 |
| | | 3000(a) | 26788 | 41442 | 59508 | 73380 | 58110 |
| | | 4000(a) | 29132 | 48433 | 63273 | 65730 | 48507 |

The critical stretching rate values provided in Table 7 are on average of the order of 10⁴ s⁻¹. For cases with higher flow rates, the dispersion of results is acceptable. In the cases of lower flow rates, these values were lower, especially for the case of 50 ml/min. This is expected, since lower flow rates allow slower passage times through the zones with high stretching rate values. The adopted model considers a step function for degradation, without taking into account the variations with Pₑ and t⁺ observed in Rognin's model. Therefore, the observed reduction of the critical value for lower flow rates is associated with this effect. Nevertheless, the values obtained with the adopted approximation are considered consistent and allow establishing a reference value to determine a priori, when selecting components, whether degradation will occur. This value obviously always depends on the characteristics of the adopted solution.

Regarding the determination of the value of Y, it was proposed, as indicated above, to be close to 4. From the results of the simulations, a conservative value appears to be Y=2,5.

Degradation tests were also carried out using the second experimental setup described above, passing each of the following solutions—2000 ppm Floopam 3631S in CS water, 2000 ppm Floopam 3631S in DB water, 2000 ppm AN125 in CS water, and 2000 ppm AN125 in DB water— through the ABAC VA125 IT valve, whose opening was adjusted to ¼ turn of the control handle from its closed position. This implies a rather narrow flow section, selected in this way to produce degradation of the solutions, while ensuring that the Deborah number values are greater than one. It is important to mention here that, given the characteristics of the available pump, the maximum possible flow rate was 200 ml/min.

With the experimental data (from the second experimental setup, passing the four 2000 ppm solutions through the valve), numerical CFD simulations were carried out on the ABAC VA125 IT valve, using the degradation model of the method of the present invention, which was implemented in the ANSYS/Fluent 19.1 software by means of a user-defined routine, and testing different values of the parameters s_{cr} and Υ that resulted in simulated degradation values acceptably close to those observed experimentally.

More than thirty-five transient simulations were performed for the different combined solutions and flow rates, proposing different values of the parameters s_{cr} and Υ until results similar to the experimental ones were obtained in terms of degradation and viscosity.

Below are presented the values of Υ (Gamma) and s_{cr} that yield the degradation percentages shown in the “% Degrad Simul” column, compared with those obtained from the experiments with the second experimental setup, where the 2000 ppm Floopam 3631S in DB water solution was not considered because of confusing data and because it fell between two extreme cases.

TABLE 8
Critical deformation rate values determined with
the second experimental setup and their corresponding
simulations and degraded mass fractions in %.

| | | | | | % | % |
|---|---|---|---|---|---|---|
| | | | S_{cr} | | Degrad | Degrad |
| Polymer | Water | Flow rate | (1/s) | Gamma | Simul | Exp |
| FLOOPAM | CS | 100 ml/min | 600 | 1.1 | 25 | 26 |
| 3631S | | 150 ml/min | 712.5 | 1.35 | 39 | 40 |
| 2000 ppm | | 200 ml/min | 800 | 1.45 | 44 | 42 |
| AN125 | CS | 100 ml/min | 500 | 2.4 | 24 | 26 |
| 2000 ppm | | 150 ml/min | 615 | 2.5 | 37.5 | 36 |
| | | 200 ml/min | 700 | 2.5 | 43 | 41 |
| AN125 | DB | 100 ml/min | 520 | 2.4 | 32.5 | 30 |
| 2000 ppm | | 150 ml/min | 530 | 2.5 | 42.5 | 42.5 |
| | | 200 ml/min | 560 | 2.5 | 61.5 | 62.5 |

It should be noted that the degree of degradation is obtained by applying the degradation criterion of the present invention, starting from approximate values of s_{cr} and Υ already theoretically established (Y approximately between 2.5 and 4, and s_{cr} on the order of hundreds for the Deborah numbers observed in the experiments with the second experimental setup).

The simulation is then performed and the degraded mass fraction obtained from the simulation is compared with the degraded mass fraction obtained from the valve tests. If they are not equal, the values of Υy s_{cr} are modified in the simulation until they are adjusted.

To obtain the degraded mass fraction in the simulation, a monitor of the degraded solution mass fraction is placed at the outlet of the simulation domain, and an average outlet value is obtained, which is compared with the experimental data. A viscosity monitor is also placed at the outlet of the domain, allowing the determination of an average outlet viscosity, where deformation rates are very low (close to zero), and the viscosity obtained in the simulation is compared with that obtained in the experiments.

This is carried out iteratively, smoothly modifying the critical parameters s_{cr} and Υ used in the simulation until comparable results between simulation and experiment are obtained.

The critical stretching rate values provided in Table 8 are on average of the order of 10² s⁻¹. The Υ values are between 1.2 and 1.45 for Floopam 3631S, and 2.5 for AN125. A slight tendency toward higher values in both cases is observed as the flow rate increases. The adopted model considers a step function for degradation, without considering the variations with Pₑ t⁺ observed in Rognin's model. In this case, it seems a reasonable assumption to consider the phenomenon of uncoiling of the polymeric macromolecules as relevant, which occurs when the deformation rates are high, even reaching breakage (mechanical degradation, which is macroscopically observed as a decrease in viscosity). The velocity values in the contraction, in the case of a capillary tube in the test of the first experimental setup, are on the order of tens of cm per second, whereas in the tests with the second experimental setup they are on the order of hundreds of cm per second.

Thus, it is expected that the cases analyzed with the second experimental setup correspond to N_{De}>>1, whereas those of the first experimental setup can be associated with cases where N_{De}<<1. This allows explaining the difference of one or more orders of magnitude between the critical deformation rate values found in both experiments.

Finally, from the previous tests, it is obtained that conservative values of Y lie between 1.2 and 2.5. For typical field cases, it is recommended to use Y of 2.5 as an initial tentative value. It can then be decreased, in which case some increase in degradation observed in the simulation would be expected.

The critical deformation rate values s_{cr} depend on the Deborah number N_{De}. For field cases, this number is typically greater than unity; therefore, in such cases, conservative values are usually in the range 500<s_{cr}<800. However, at typical field injection flow rates (on the order of 100 m³/day), it is recommended to adopt the higher end of the range, s_{cr}=800. For the purpose of testing the sensitivity of the model, it has been found from experience in the simulations carried out in this work that the value of this critical deformation rate, in real field cases, can be considered up to values of s_{cr} equal to 3000, and it is not advisable to exceed such value.

For laboratory cases where the number N_{De} may typically be less than one, it can then be considered that 30000<s_{cr}<70000 using Table 7 of this report as a reference depending on the flow rate and the type of solution.

For the polymer solution of interest, experimentally the critical deformation value at a given temperature s_{cr} can be determined from the knowledge of the volume fraction of solution degraded as a result of passing through a system with a capillary. This system may be the one described in API RP 63. Knowing the degraded fraction and through numerical simulations for the geometry of the considered capillary system and the flow rate at which the test was performed, s_{cr} can be established as explained above.

The knowledge of the degraded fraction of the polymer solution can be determined taking into account the methodology described in the articles [E. Perez et al Sci Reports, 2024-E Perez et al Journal of Petroleum Science and Engineering 2022, E Perez et al SPE 2025 in Press]. This methodology requires only the determination, at a single temperature, of the viscosity value at a single shear rate of the original and degraded polymer solution after capillary passage. The viscosity measurement can be performed with traditional viscometers such as the Brookfield.

1. A method for determining the degree of degradation of a polymer solution comprising a polymer dissolved in water with a certain salinity and used in an enhanced oil recovery (EOR) process, said method comprising the following steps:
a) determining the critical deformation rate s_{cr} and the dimensionless parameter Υ, which is a critical value related to the transit time of the solution particles through regions where deformation rates could cause degradation, in order to determine the degree of degradation of the polymer solution based on these two critical parameters; and
b) comparing the effective local deformation rates occurring in the flow within a given flow fitting with the critical deformation rate s_{cr} and the parameter Υ, in order to determine whether degradation occurs in any region of the flow in a computational fluid dynamics simulation.

2. The method according to claim 1 further comprises, prior to step a), a step of determining the rheology of the non-degraded solution and of the fully degraded solution, wherein the rheology of both the non-degraded solution and the fully degraded solution is determined using the equation:
$$η = {η}_{∞} + ( {η}_{0} - {η}_{∞} ) {( 1 + {( λ \dot{γ} )}^{a} )}^{\frac{( n - 1 )}{a}}$$
where η is the viscosity at a deformation rate \dot{y}, η_{∞}, is defined as the viscosity at an infinite shear rate, η₀ is the viscosity at zero shear rate, λ is a shear relaxation time or characteristic shear time, n is the power-law index, and a is a dimensionless parameter describing the transition from the zero-shear-rate region to the power-law region.

3. The method according to claim 1, wherein the critical deformation rate s_{cr} in step a) is obtained through the following sub-steps:
a-i) determining a principal eigenvalue field of the deformation rate tensor, selecting from said eigenvalues the value that is positive and has the highest magnitude s_{I} (first principal eigenvalue of the rate (or velocity) of deformation tensor);
a-ii) determining the streamlines of the flow;
a-iii) determining an extreme streamline that carries the degraded solution; and
a-iv) determining the maximum principal eigenvalues along that line that produces degradation; and
a-v) assigning a maximum value of the maximum principal eigenvalues as the critical deformation rate s_{cr}.

4. The method according to claim 1, wherein conservative values of Y are determined between 1.2 and 2.5.

5. The method according to claim 4, wherein a value of Y equal to 2.5 is used as a first tentative value for typical field cases.

6. The method according to claim 1, wherein conservative values of the critical deformation rate s_{cr} are used in the range 500<s_{cr}<3000 for Deborah numbers N_{De}, greater than 1.

7. The method according to claim 6, wherein conservative values of the critical deformation rate s_{cr} are used in the range 500<s_{cr}<800.

8. The method according to claim 1, wherein conservative values of the critical deformation rate s_{cr} are used in the range 30000<s_{cr}<70000 for Deborah numbers N_{De} less than 1.

9. The method according to claim 1, wherein the simulation comprises calculating the first principal eigenvalue of the tensor s_{I} and the rotation period, point by point within the flow, and comparing the values of s_{I} and t_{w} respectively with the critical values s_{cr} and Υ, wherein degradation generally occurs when the first principal eigenvalue of the deformation rate tensor s_{I} exceeds the critical value s_{cr}, that is:
$${s}_{I} \geq {s}_{c r}$$
and, at the same time:
$${s}_{I} {t}_{w} \geq ϒ .$$

10. The method according to claim 1, wherein the simulation comprises calculating the first principal eigenvalue of the tensor s_{I} and the transit time tₜᵣₐₙₛ, point by point within the flow, and comparing the values of s_{cr} and Υ respectively with the critical values s_{I} and tₜᵣₐₙₛ, wherein degradation generally occurs when the first principal eigenvalue of the deformation rate tensor s_{I} exceeds the critical value s_{cr}, that is:
$${s}_{I} \geq {s}_{c r}$$
and, at the same time:
$${s}_{I} {t}_{trans} \geq ϒ .$$
